(19) United States
(12) Patent Application Publication
(10) Pub. No.: US 2021/0214675 A1
(43) Pub. Date: Jul. 15, 2021

(54) Method of Producing High-Strength Composite Sheet Composed of Fiber-Reinforced Grown Biological Matrix

(71) Applicant: Moshe Sergei Buxbaum Kruger, Teaneck, NJ (US)

(72) Inventor: Moshe Sergei Buxbaum Kruger, Teaneck, NJ (US)

(21) Appl. No.: 16/697,418

(22) Filed: Nov. 27, 2019

(51) Int. Cl. C12N 1/20; C09D 1/00; C12N 1/16; C09D 5/24

(52) U.S. Cl. CPC C12N 1/20; C09D 1/00; C12N 2533/00; C09D 5/24; C12N 1/16

(57) ABSTRACT

The present invention discloses a method of forming high-strength composite sheets composed of a fiber-reinforced biological matrix. This invention discloses three aspects of technology: fiber placement, biological matrix growth, and material insertion within a biological matrix. A cell culture is grown in a tank of nutrients. The cells form a skin and can envelope fibers that are positioned at a specific location. When taken out of its solution, it dries pinning the fibers into the sheet to form a lightweight, waterproof layer. By creating mostly axial fibers and rolling them into a tube, extremely strong structural cylinders can be formed. By incorporating a mesh of fibers (woven or knit) an extremely tough layer is formed that can be stacked and made into ballistic armor. The addition of foreign additives; graphite, metal powder may increase the strength, performance, and conductivity of the material.

### BACKGROUND

The strongest, lightest material currently available for building parts is carbon fiber, which is comprised of strong but brittle carbon fibers reinforcing a plastic (typically epoxy) matrix. Carbon fiber is very expensive for two reasons. First it is made from a polymer, and more than 50 percent is lost in the process. Second, for the carbon to shed the acrylic, two processes require hundreds of degrees Celsius to materialize plus the additional labor of weaving the fibers evenly. Carbon fiber is used in high-strength, ultra-light applications such as high-end bicycle frames, tennis rackets, race cars, and spacecraft.

Slightly heavier, and not as stiff but much tougher is aramid (Kevlar). Aramid is fire-resistant, and can absorb much more shock without breaking. Aramid is used in applications requiring impact resistance such as ballistic armor. Some fabrics are available with a combination of the two fibers.

Fiberglass which is produced from glass is considerably heavier and weaker. Fiberglass reinforced polymers are commonly used in mass-produced boat hulls. Efforts are ongoing to produce volumes of bioengineered spider silk which is similar to Kevlar, and with some species substantially greater in strength and toughness. Other natural fibers such as hemp and jute have also been proposed for fiber reinforced ballistic armor and could be used to reduce cost and environmental impact.

There are a number of plastics commonly used as the matrix in which reinforcing fibers are placed, including polyester, vinylester, and epoxy. The strongest is epoxy, which is also the most expensive, the most difficult to work with, and environmentally toxic, but all the thermoset plastics are toxic and emit volatile organic compounds (VOC) while curing.

Composite materials are formed by layers of woven fibers, impregnated with resin, formed with manual labor and for highest strength, compressed in a vacuum mold using external air pressure to form the part. The process is quite labor-intensive for high-quality parts.

Ballistic armor is a protective garment that is worn to prevent injury from either stabbing, slashing, or a penetrating high velocity projectile. There are two main types of ballistic armour; soft and hard-plate. The majority of non-plated armour is made from multiple layers of aramid sewn together and covered in cloth. Early plates were made of steel, but this is extremely heavy and difficult for soldiers to wear. Today, most hard-plated armour consists of a ceramic composite material (boron carbide or silicon carbide) backed by ultra-high molecular weight polyethylene inserted into a pocket of soft armor. There are also experimental armors containing non-Newtonian liquids which become solid when impacted, shedding energy.

A recently-discovered problem facing soldiers is the effect of an Electromagnetic Pulse (EMP). When an explosion occurs, in addition to the physical blast there is a wave of electrons liberated by the explosion. When the brain and body are at slightly different distances from the blast, a small current is induced in the spine causing brain damage. It has been estimated that 80% of brain damage incurred from a survivable bomb blast is physical and 20% from the EMP. In order to eliminate the current, shielding must create a Faraday cage enclosing the body and head electromagnetically so that current goes around the body, instead of through the spine.

Manufacturers and armies around the world are continually trying to improve ballistic armor to reduce the damage from bullets, and at the same time trying to create new weapons to defeat current armor. The limiting factor is the ability of soldiers to bear the weight of the armor, which can reduce combat effectiveness through exhaustion.

### BRIEF SUMMARY OF THE INVENTION

This invention is a new method of constructing advanced composite materials that are environmentally benign. A composite material consists of a matrix containing fiber-reinforcement.

This method uses a cell culture to create a matrix in which fibers are embedded. A SCOBY (Symbiotic Colony of Bacteria and Yeast) is one example of such a cell culture. It is formed by bacteria and yeast in a solution of tea and sugar. The SCOBY is produced by an anaerobic formation of ethanol and acetic acid, followed by the oxidation of sugar to lactic acid, then to acetate, forming a cellulose biological film with a texture similar to that of leather. Alternatively, the matrix may be a culture of plant cells, animal cells, or some combination.

The matrix may be reinforced using fibers such as carbon fiber, aramid, spider silk, jute, hemp, or other artificial or natural fibers. The fibers are stronger in tension and compression than the matrix. The novel aspect of this invention is the method of binding the fibers. Instead of embedding fibers into liquid plastic which solidifies, a biological matrix is grown around the fibers.

Fiber is stretched on a frame and placed in a mixture of either vinegar, kombucha, or another culture at a certain height so that a biological film envelops the fibers thus locking the pre-tensioned fiber into the material while affixing the fibers in place. This method of growing the material is inexpensive compared to current processes, scalable to large industrial quantities, and takes only 2-3 weeks to be ready for harvest. There are also ways of accelerating growth such as having a pre-formed ‘starter’ biofilm which, when placed into the solution, will give a boost to the formation of the film. Further, by having a steady feed of sugar and caffeine the layer growth can be accelerated.

Automated culturing of fiber-reinforced materials can create an advanced composite that is extremely strong, tough, with desired physical characteristics tailored by the use of the reinforcing fibers (axial strength, uniform tensile strength, penetration resistance). It is far less expensive than current methods of laying up layers of fiber-reinforced plastics and is more environmentally benign. The growth of the biological film is substantially less detrimental to the environment than gluing layers of cloth together. There are no Volatile Organic Compounds produced, no energy is required to heat the material other than maintaining a stable temperature in the tank for growth.

To recycle the material, the biological matrix can be composted either naturally or artificially, and the remaining internal fibers can be recycled if they are not biodegradable, and or reused by reweaving them if they have been undone. After recycling a biological matrix can be regrown around the fibers.

A long sheet of material can be grown, limited only by the length of the reinforcing fibers and the tank. A roll to roll system can pull an even longer sheet limited only by the length of available fiber-reinforcement.

### DRAWINGS

FIG. 1 illustrates a tank containing SCOBY with a frame holding pre-tensioned aramid fibers.

FIG. 2 illustrates a batch system in which the sheet is grown with aramid fibers and then rolled onto a spool.

FIG. 3 illustrates a roll-to-roll system in which the fiber reinforcement is continuously pulled through the tank.

FIG. 4 illustrates a system for washing, drying, and collecting the composite sheet on a spool.

FIG. 5 illustrates a second tank where nutrients and conductive material is added to the surface.

### DETAILED DESCRIPTION AND BEST MODE OF IMPLEMENTATION

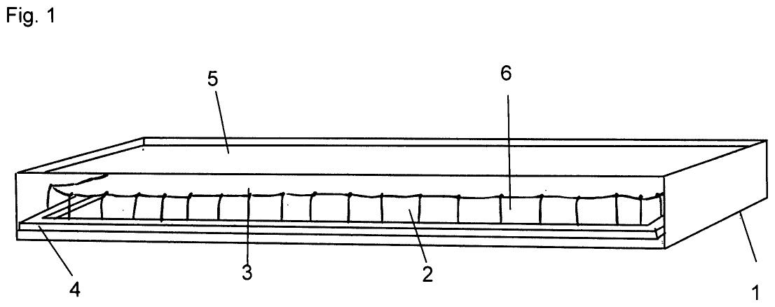

FIG. 1 shows a tank 1 filled with a nutrient solution and cell culture 6, with a biological film 5 growing near the surface, with fibers 3 stretched on a frame 4 using hooks 2 to maintain the tension of the fiber reinforcement. Because it is under tension, the resulting sheet will have low strain (will not dramatically stretch under load).

Fibers can be spun into thicker ropes with most of the fiber in the axial direction and fewer across to create structural material with strength mostly in the axial direction.

Fiber reinforcement can be woven using a loom with varying spacing of fibers in both warp and weft directions to precisely control strength in a plane. Fiber can be knit, creating loops that can stretch and absorb impact. Fiber can be crocheted, using only one strand, to create a structure that is three dimensional allowing connections between layers via the stronger fibers rather than only the dried cell material. This should make the material more resistant to delamination and also the energy of impacts may be more diffused.

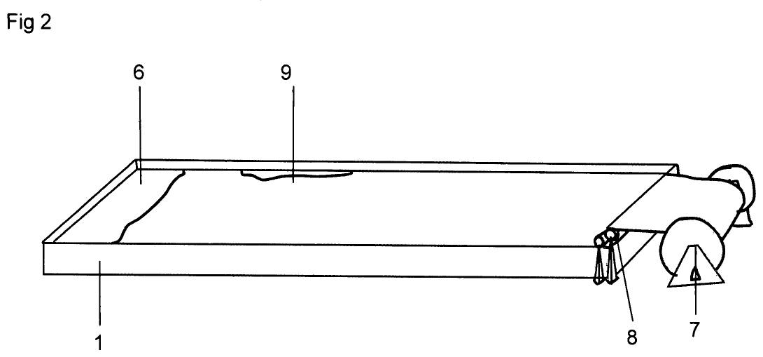

FIG. 2 shows a batch system in which a tank 1 contains nutrients 6 and cell culture with embedded fiber 10 being lifted out of the tank with rollers 8,9 and rolled onto a spool 7.

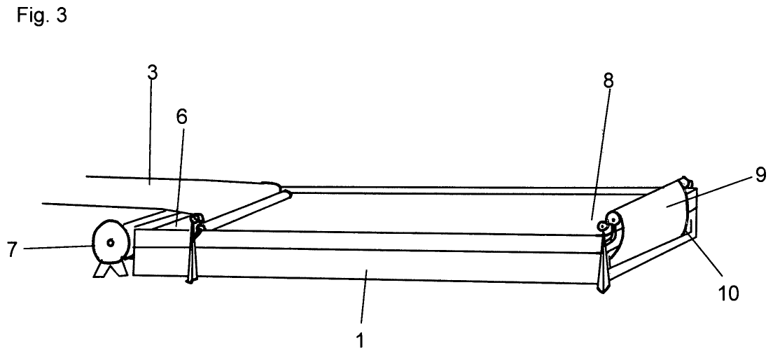

FIG. 3 shows a tank 1 containing nutrient solution 6 with incoming fiber reinforcement 3 tensioned and moved by rollers 8, outgoing biological composite with fiber reinforcement 9, going into final processing cleaning and drying (10). The dried sheet is collected on the receiving spool 7.

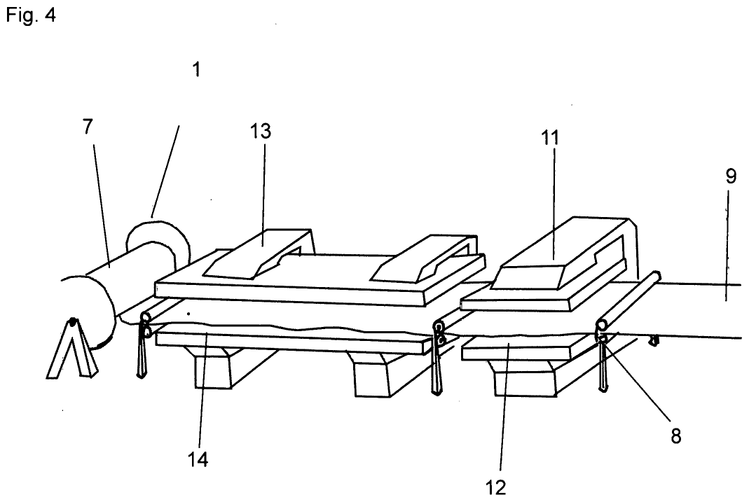

FIG. 4 shows the hidden details of the previous process 10 (cleaning and drying). Wet biological composite with fiber reinforcement 9 comes through the rollers 8 to be washed with the water and cleaning solution supply 11 using the water and cleaning solution dispenser 12. The cleaned biological matrix with fiber reinforcement is then passed through the drying unit comprised of the heating source 14 and the heating supply 13. The dried biological matrix with fiber reinforcement is then collected on the receiving spool 7.

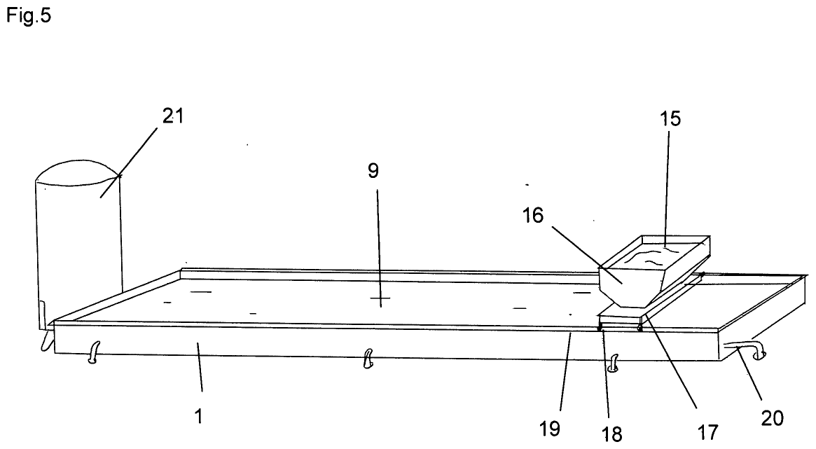

FIG. 5 shows the dispensing conductive material/powder 15 which is held in the conductive material/powder holder 16 which is then programmably dispersed by the conductive material/powder dispenser 17. The conductive material/powder holder 16 runs on rails 19 on wheels 18 along the edge of the tank 1. The nutrient solution tank 21 disperses nutrient evenly through nutrient/solution supply pipes 20.

### REFERENCE NUMBERS

1. Tank
2. Tensioning Hooks
3. Fiber
4. Frame
5. Biological composite
6. Nutrient Solution for biological growth
7. Receiving Spool
8. Rollers
9. Biological composite with fiber reinforcement
10. Opening towards final processing, cleaning, drying.
11. Water and cleaning solution supply
12. Water and cleaning solution dispenser
13. Electrical or gas supply for heating/drying.
14. Heating source
15. Conductive material/powder
16. Conductive material/powder holder
17. Conductive material/powder dispenser
18. Wheels
19. Rails
20. Nutrient/solution supply pipes
21. Nutrient/solution tank

What is being claimed is:

1. A fiber-reinforced biological material (biological matrix) comprising:
a. a cell culture in a solution that can be grown into a single sheet of biological matrix;
b. a tank containing the cell culture capable of holding fiber reinforcement at an exact position so that it is encapsulated into the growing biological matrix;
c. fiber-reinforcement that is inserted in a rack maintaining tension to maximize the strength of the sheet;
d. fibers that are compressed into a plane as the biological matrix dries and shrinks in the vertical dimension;
e. strengthened biological matrix as a result of the addition of particles to the cell culture that are incorporated into the biological matrix;
f. a strong insulating layer once the biological matrix is dried;
g. a conductive layer with the addition of conductive materials that reduce the resistivity of the biological matrix;
h. a controlled growth period, followed by drying, allowing precise control of the thickness of the resulting dried sheet of biological matrix, including extremely thin sheets with a very high ratio of fiber to biological matrix;
i. protection of the sheet from environmental degradation by gluing it in layers and coating with a protective chemical or glue;
j. the creation of a mold of a desired shape, applying a chemical that allows the biological matrix to release easily; and
k. a multi-layer composite material composed of layers of biological matrix each enveloping fiber reinforcement, and bonded by adhesive.

2. The method of claim 1 wherein the cell culture is a Symbiotic Colony of Bacteria and Yeast (SCOBY).

3. The method of claim 1 wherein the cell culture is a bacterial colony.

4. The method of claim 1 wherein the cell culture is a culture of plant cells or animal cells.

5. The method of claim 1 where the fiber reinforcement is high-modulus artificial fibers such as aramid, carbon fiber, or fiberglass.

6. The method of claim 1 where the fiber reinforcement is natural fibers such as jute or hemp.

7. The method of claim 1 where the fiber reinforcement is a tough material such as spider silk, which can be produced by genetically modified organisms.

8. The method of claim 1 where the density of the biological matrix is decreased, and the strength increased, by incorporating light, strong particles such as graphite, graphene, or carbon nanotubes into the solution so that they are incorporated by the cell culture into the biological matrix.

9. The method of claim 1 where graphene paint (conductive coating) is applied after the biological matrix is dried to create a conductive surface useful for electromagnetic shielding or to connect embedded circuitry to the biological matrix; the solvents can kill the biological cells so the paint must be applied either post-processing in a separate tank or at low concentrations so that the culture can survive and continue to grow around the conductive coating;

10. The method of claim 1 where metals such as silver and copper are added to the surface to create a conductive coating useful as electromagnetic shielding; since these materials are toxic to the biological matrix, they must be added in a separate tank.

11. A means of growing the fiber-reinforced biological matrix comprising:
a. a tank holding nutrients and cells being grown into a biological matrix;
b. a rack holding reinforcing fibers in tension in the cell culture while the biological matrix grows around them;
c. a mechanism to lift the rack in and out of the tank;
d. a roll-to-roll mechanism to grow infinite-length sheets of biological matrix by slowly drawing them through a long tank while maintaining tension;
e. a washing mechanism to remove the remaining live cells and slime from the surface of the biological matrix;
f. a drying mechanism for controlled rate of drying under tension so the biological matrix has a precise, predictable dimension;
g. a boiler to create a sterile solution to feed the growing biological matrix;
h. rollers that can compress and join multiple layers of biological matrix into a thicker sheet;
i. a hydroponic system to deliver nutrients such as caffeine, sugar, or any other chemical or organic compound to increase the rate of growth or quality of the biological matrix;
j. a sprayer to evenly distribute solutions containing additives on the surface or from beneath to be incorporated into the biological matrix; and
k. a sterilization system so that no unwanted bacteria form around the biological matrix.
